(19) United States
(12) Patent Application Publication
(10) Pub. No.: US 2026/0325802 A1
(43) Pub. Date: Oct. 22, 2026

(54) MEDICAL APPARATUS WITH INFLATABLE MEDICAL BALLOON FOR ENDOLUMINAL USE HAVING INDEPENDENTLY VARIABLE DIAMETER PORTIONS AND RELATED METHODS

(71) Applicant: CLEARSTREAM TECHNOLOGIES LIMITED, Enniscorthy, County Wexford (IE)

(72) Inventors: Artur Spodobalski, Enniscorthy, County Wexford (IE); Michael Whelan, Enniscorthy, County Wexford (IE); Christopher J. Cosgrave, Enniscorthy, County Wexford (IE); Keith Bates, Enniscorthy, County Wexford (IE)

(21) Appl. No.: 19/180,453

(22) Filed: Apr. 16, 2025

(51) Int. Cl. A61M 25/10; A61F 2/958

(52) U.S. Cl. CPC A61M 25/1002; A61F 2/958; A61M 2025/1059; A61M 2025/1061; A61M 2025/1072; A61M 2205/3331; A61M 2210/12

(57) ABSTRACT

A medical apparatus includes a variable diameter balloon for endoluminal use. The balloon may have an hourglass shape when inflated, with at least two spaced portions adapted to expand independently upon inflation. Specifically, the balloon includes a first portion expandable to a first diameter, a second portion expandable to a second diameter equal to or greater than the first diameter, and a third portion of the balloon between the first and second portions having a third diameter less than the first diameter or the second diameter when the balloon is inflated, thus providing the hourglass shape. The balloon may be carried by a shaft including a first inflation lumen in communication with a first opening associated with the first portion of the inflatable balloon and a second inflation in communication with the second portion of the inflatable balloon, with each lumen associated with different proximal and distal ports within the first and second portions of the balloon. The medical apparatus may also include an implant, such as a stent.

### BACKGROUND

Medical apparatuses in the form of balloon catheters have been devised for use in various medical procedures, including angioplasty and balloon expandable stent/stent graft (implant) delivery. Commonly, a guidewire introduced percutaneously into the patient's vascular system advances via steering to the site of a stenosis. A dilatation balloon on the catheter is advanced over the guide wire until the balloon is positioned within the stenosis (which makes it desirable to provide the balloon with a particularly low profile, yet with adequate strength to be pushed through the vasculature). Upon inflation, the balloon compresses the stenosis by dilatation of the blood vessel to re-establish a more adequate blood flow path past the stenosis. To facilitate even compression pressure distribution along the length of the stenosed lesion, it is a clinical preference that the dilation balloon be sized and centered relative to the stenosis so as to fully engage the lesion.

Balloon dilation catheters have also been utilized in balloon expandable implant delivery in which the implant is disposed about the balloon and inflated into place at the stenosis. Catheter operators seek accurate deployment of the implant directly on the diseased tissue of the vessel in order to avoid stent migration to either side of the diseased tissue thereby avoiding or minimizing the chance of leaving some of the diseased tissue untreated. Accurate deployment also desirably avoids adversely affecting healthy tissue.

Implant misplacements may occur because of specific inflation dynamics experienced by the expandable balloon when deploying the implant. Many balloon expandable implant delivery catheters inflate the balloon preferentially from the proximal end of the balloon (and may suffer from the inability to transmit inflation fluid from the proximal to the distal end as a result of the placement of the compressed or unexpanded implant over the balloon). During inflation, the expanding balloon may form an asymmetrical growth or inflation wave that may be said to drive or plow the implant so that it opens progressively from one end to the other along the front of the inflation wave. The wave may sometimes cause the implant to disengage prematurely from the balloon, and may also cause a deploying implant to displace longitudinally away from its intended delivery site, thereby potentially ineffectively treating the diseased lesion within the patient's vasculature. This premature deployment is often described as “watermelon seeding.” Accuracy of positioning is also important for stents and stent grafts, as missing the target can have deleterious consequences.

Balloons may also be useful in performing procedures in vessels having variable diameters, such as for instance the coronary sinus. Yet, current approaches to balloon catheters do not account for this possibility, since typically a single balloon is provided having a single inflation port, or sometimes multiple ports supplied from a common inflation lumen, thus achieving simultaneous inflation of different portions of the balloon at the same rate. Once inflated, the resulting balloon has a generally uniform maximum diameter, and thus may in some circumstances be unable to provide effective treatment in variable diameter vessels.

Accordingly, a need is identified for a balloon catheter that may be inflated in a preferential manner to achieve a variable diameter, and which may also achieve a more controlled regulation of inflation in situ to facilitate the proper delivery of an implant, such as a stent, stent graft, or the like.

### SUMMARY

An object of the disclosure is to provide a medical apparatus for endoluminal use with a medical balloon having an independently variable diameter to allow for effective treatment in vessels of varying diameters. The apparatus may also be used in connection with an implant, such as a stent, stent graft, or the like, to achieve delivery in a highly controlled, predictable and effective manner.

According to a first aspect of the disclosure, a medical apparatus includes a balloon having an hourglass shape when inflated, the balloon including at least two spaced portions adapted to expand independently during inflation of the balloon.

In one embodiment, the at least two spaced portions include a first portion expandable to a first diameter and a second portion expandable to a second diameter equal to or greater than the first diameter. The balloon, which may be compliant or semi-compliant, further includes a third portion between the first and second portions, the third portion having a diameter less than the first diameter or the second diameter when the balloon is inflated. The third portion of the balloon has a third diameter less than the first diameter or the second diameter. The third portion of the balloon may be connected or attached to the shaft to block fluid communication between the first portion of the balloon and the second portion of the balloon.

The apparatus may further include a shaft including a first inflation lumen in communication with a first opening associated with the first portion of the inflatable balloon and a second inflation in communication with the second portion of the inflatable balloon. A first port is provided in communication with the first lumen and a second port in communication with the second inflation lumen. The first and second ports are adapted to be provided with an inflation fluid independently or simultaneously.

According to a further aspect of the disclosure, a medical apparatus is provided including an inflatable balloon including a first portion expandable to a first diameter, a second portion expandable to a second diameter, and a third portion between the first and second portions, the third portion having a third diameter less than the first diameter or the second diameter. A shaft is also provided for supporting the inflatable balloon, the shaft including a first inflation lumen in communication with a first opening associated with the first portion of the inflatable balloon and a second inflation lumen in communication with the second portion of the inflatable balloon. A first connector is adapted for supplying fluid to one or both of the first and second inflation lumens.

In one embodiment, the third portion of the balloon, which may be compliant or semi-compliant, is attached to the shaft to block fluid communication between the first portion of the balloon and the second portion of the balloon. The first connector is adapted for supplying fluid to the first inflation lumen and further including a second connector adapted for supplying fluid to the second inflation lumen. Alternatively, the first connector may be in communication with a third inflation lumen in communication with the first and second inflation lumens, and further including a valve for controlling fluid flow from the third inflation lumen to the first and second inflation lumens.

This disclosure also pertains to a method of inflating a medical balloon inserted in a vessel. The method comprises inflating spaced apart portions of the medical balloon while restraining an intermediate portion of the medical balloon to provide the inflated medical balloon with an hourglass shape within the vessel.

In one example, the inflating step comprises supplying inflation fluid simultaneously to first and second end portions of the medical balloon. In another example, the inflating step comprises supplying inflation fluid independently to first and second end portions of the medical balloon. The method may further comprise the step of delivering an implant to the vessel via the medical balloon.

### BRIEF DESCRIPTION OF THE DRAWINGS

The above and further advantages of the disclosure may be better understood by referring to the following description in conjunction with the accompanying drawings in which:

FIG. 1 is a side view of one embodiment of the medical apparatus according to the disclosure.

FIG. 2 is an enlarged side view of the medical apparatus of FIG. 1.

FIG. 3 is a cross-sectional view of the medical apparatus taken along line 3-3 of FIG. 1.

FIG. 4 illustrates a possible use of the medical apparatus in a vessel or other body lumen.

FIGS. 5, 6, 7, and 8 illustrate a possible sequence of using the medical apparatus.

FIG. 9 schematically illustrates an alternative embodiment of the medical apparatus.

The dimensions of some of the elements in the drawings may be exaggerated relative to other elements for clarity or several physical components may be included in one or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the disclosure in relation to various inventions. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination. The description provided below and in regard to the figures applies to all embodiments unless noted otherwise, and features common to each embodiment are similarly shown and numbered to the greatest extent possible to lend clarity, but should not be considered limiting in any respect.

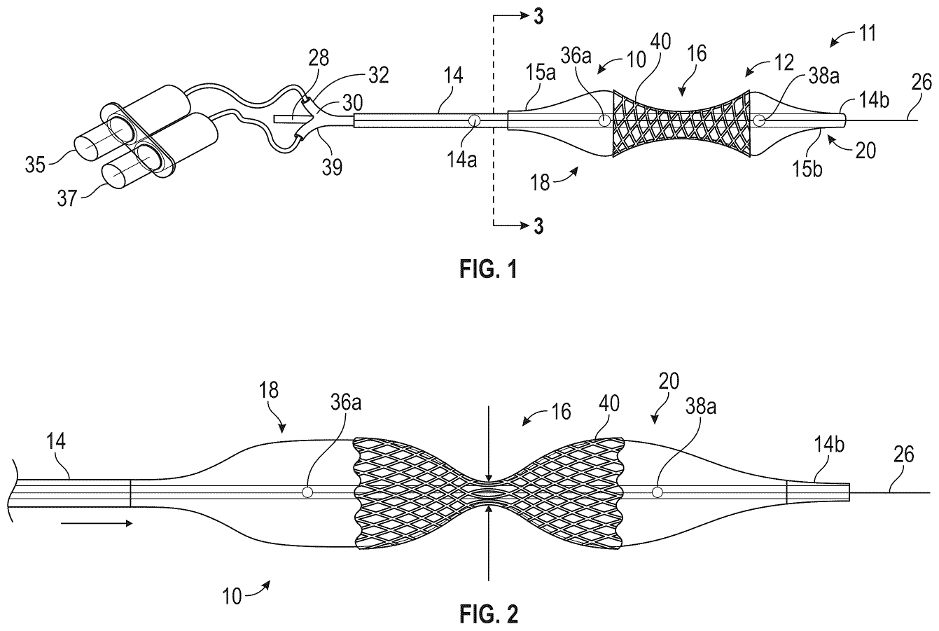

With reference to FIG. 1, provided is a catheter 10 having a distal end portion 11 with a balloon 12 mounted on a catheter shaft 14. The balloon 12 has an intermediate portion 16 bounded by spaced apart proximal and distal end portions 18, 20. The intermediate portion 16 may be connected to the shaft 14, such as by an adhesive, band, or other retainer, to substantially obstruct fluid flow between the proximal and distal end portions 18, 20 of the balloon 12, and thus providing the balloon with independent interior compartments within these portions. The balloon 12 is also sealed to the shaft 14 at the proximal end 15a and distal end 15b of the end portions 18, 20, which allows for the balloon 12 to be inflated in a manner that will be described in further detail in the following discussion.

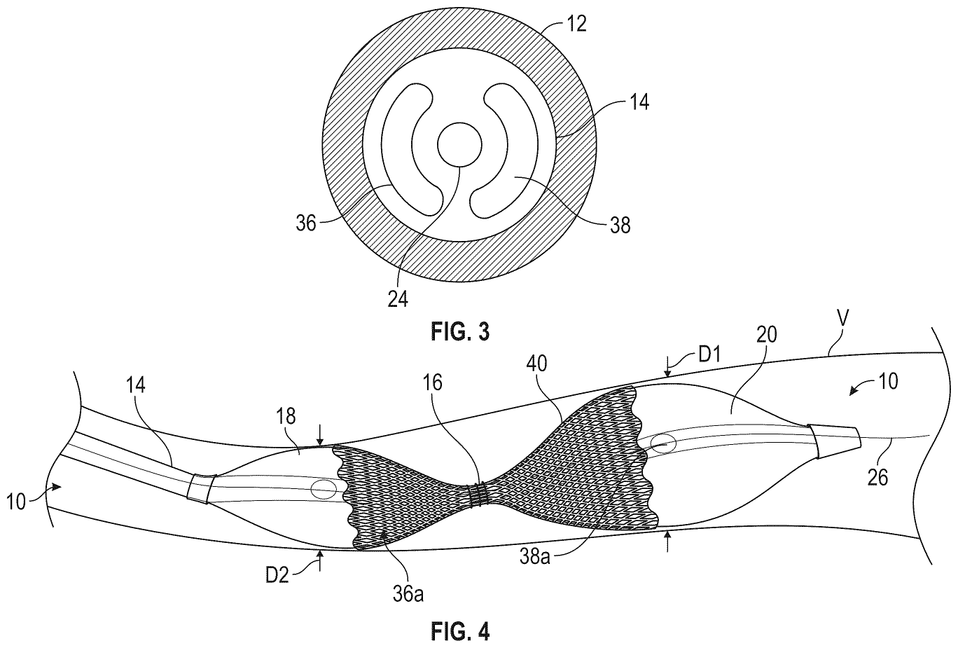

With reference to FIGS. 2 and 3, the catheter shaft 14 may include a guidewire lumen 24 that directs a guidewire 26 through the catheter 10, and along the distal end portion of which the balloon 12 may be located. As illustrated in FIG. 2, this guidewire 26 may extend through the proximal end portion of the catheter 10 via a first proximal port 28 of a connector or hub 30 into the lumen 24 to achieve an “over the wire” (OTW) arrangement. Alternatively, the guidewire 26 could also be provided in a “rapid exchange” (RX) configuration, in which the guidewire 26 exits a lateral opening 14a closer to the distal end but proximal of the balloon 12, or else may be fed through the shaft 14 near the tip 14b at a passage distally of the balloon 12 (“short” RX; not shown).

Multiple inflation ports 32, 34 may also be associated with catheter 10, such as by way of connector or hub 30, for introducing a fluid (e.g., saline, a contrast agent, or both) into the balloon 12. Specifically, each port 32, 34 is associated with a separate lumen 36, 38 formed in the shaft 14. Each lumen is also associated with a connector 35, 37 for connecting with one or more sources of pressurized fluid (such as a conventional indeflator (see FIGS. 6-8) used in connection with medical balloons). The first inflation lumen 36 is associated with a first port 36a in the first end portion 18 of the balloon, while the second inflation lumen 38 is associated with a second port 38a in the second end portion of the balloon 20. Thus, in view of the flow restriction created by the intermediate portion 16, the balloon 12 is provided with an hourglass or dog-bone shape upon inflation.

The balloon 12 may be made of a compliant or semi-compliant material, such as for example an elastic or semi-elastic polymer material. As a consequence, the continued addition of inflation fluid may result in the continued expansion of the balloon 12, yet in a manner that is controlled to avoid over-inflation that could result in a potential rupture. In light of the provision of independent inflation lumens, it can be appreciated that this feature allows for the different end portions 18, 20 of the balloon 12 to be inflated at different amounts, rates, or times to achieve different diameters for effecting different treatment, functions, or results. The balloon 12 may be fabricated of a single unitary piece of tubular material, such as using conventional forming techniques (e.g. extrusion or molding), and may have suitable dimensions dependent upon the end use to which it is put (e. g, a balloon with a larger nominal diameter and length for use in a larger vessel, and a smaller nominal diameter/length one for a smaller vessel).

Thus, as shown in FIG. 4, in a vessel V having a variable diameter, the distal end portion 20 of the balloon 12 may be inflated to a first diameter D1 using the second port 34. Likewise, the proximal end portion 18 of the balloon 12 may be inflated to a second, lesser diameter D2 using the first port 32. These different diameters D1, D2 may correspond to different diameters of the vessel V in which the balloon catheter 10 is inserted during a medical procedure to achieve a desired treatment effect.

As indicated in the drawing figures, the catheter 10 may also be provided with a payload in the form of an implant, such as an expandable stent 40. The stent 40 may be crimped onto the balloon 12 prior to inflation. Inflation in the manner described above thus provides the stent 40 with a corresponding hourglass shape. The differential inflation may also be used to regulate the position of the stent 40, for example by inflating the proximal end portion 18 of the balloon 12 initially to urge the stent 40 distally, before inflating the distal end portion 20 of the balloon 12, or vice-versa. This may be used to allow to expand the stent 40 to variable diameters along its length, and also to deploy it in a highly controlled predictable matter, including by varying the expansion of the ends of the stent independently.

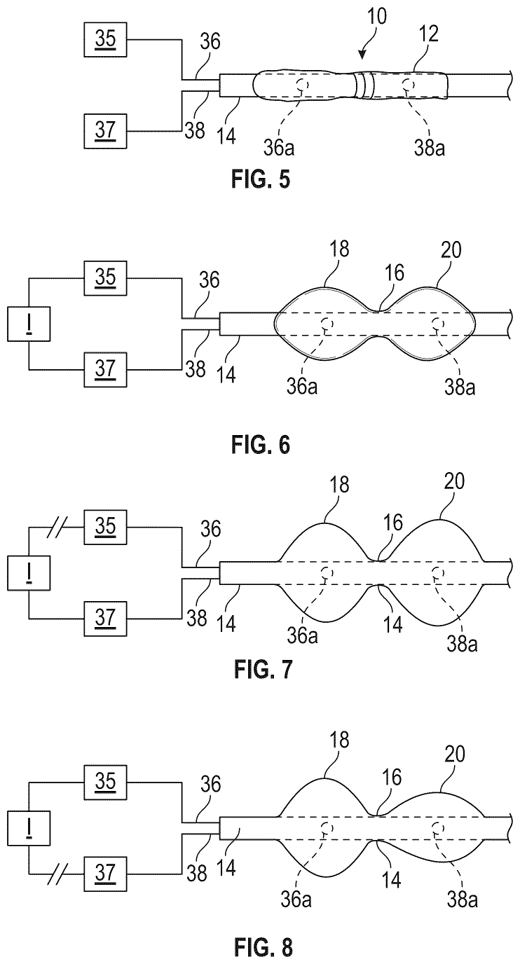

FIGS. 5-8 illustrate an exemplary sequence of operation of the catheter 10 according to one aspect of the disclosure. Initially, the catheter 10 includes a deflated folded balloon 12. Initially, an inflator I may be connected to the connectors 35, 37 to concurrently fill the end portions 18, 20 of the balloon 12 with an inflation fluid, such as to reach a nominal diameter, while the intermediate portion 16 remains secured to the shaft 14. The inflator I may then be disconnected from one connector, such as connector 35 associated with the lumen 36 in communication with the proximal end portion 18 of the balloon 12. Thereafter, fluid may be supplied only to the distal end portion 20 via the connector 37 associated with lumen 38, thus causing it to increase in diameter relative to the proximal end portion 18 of the balloon 12. Alternatively or thereafter, the inflator I may be disconnected from connector 37, such that fluid is supplied only to the connector 35 associated with the lumen 38 corresponding to the proximal end portion 18 of the balloon 12, to thus cause it to increase in diameter relative to the distal end portion 20.

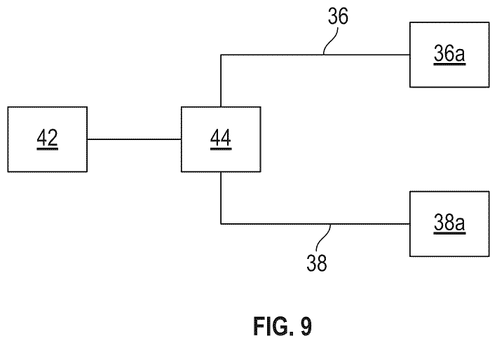

In an alternative configuration, the catheter 10 may comprise a single inlet 42 that is branched off to a distal inflation port 36a and a proximal inflation port 38b, as shown in FIG. 9. Both of these ports 36a, 36b can be closed/opened via a valve, such as a stopcock 44. This provides the user the option to have independent control of the inflation of the distal or proximal end portions 18, 20 of the balloon 12, respectively, or both may be open to inflate these portions of balloon 12 uniformly. In practice, inflation of the proximal and distal portions 18, 20 of balloon 12 may occur uniformly to a point, followed by an adjustment to allow for independent inflation of the distal and proximal end portions.

As used herein, the following terms have the following meanings: “A”, “an”, and “the” as used herein refers to both singular and plural referents unless the context clearly dictates otherwise. By way of example, “a compartment” refers to one or more than one compartment. “About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which such modifiers refer is itself also specifically disclosed. “Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A medical apparatus, comprising:
a balloon having an hourglass shape when inflated, the balloon including at least two spaced portions adapted to expand independently during inflation of the balloon.

2. The medical according to claim 1, wherein the at least two spaced portions of the balloon include a first portion expandable to a first diameter and a second portion expandable to a second diameter equal to or greater than the first diameter.

3. The medical apparatus of claim 2, the balloon further including a third portion between the first and second portions, the third portion having a diameter less than the first diameter or the second diameter when the balloon is inflated.

4. The medical apparatus of claim 3, further including a shaft including a first inflation lumen in communication with a first opening associated with the first portion of the inflatable balloon and a second inflation in communication with the second portion of the inflatable balloon.

5. The medical apparatus of claim 4, further including a first port in communication with the first lumen and a second port in communication with the second inflation lumen.

6. The medical apparatus of claim 5, wherein the first and second ports are adapted to be provided with an inflation fluid independently or simultaneously.

7. The medical apparatus of claim 5, wherein the third portion of the balloon is attached to the shaft to block fluid communication between the first portion of the balloon and the second portion of the balloon.

8. The medical apparatus of claim 1, wherein the balloon is compliant or semi-compliant.

9. An implant in combination with the medical apparatus of claim 1, the implant having an hourglass shape.

10. A medical apparatus, comprising:
an inflatable balloon including a first portion expandable to a first diameter, a second portion expandable to a second diameter, and a third portion between the first and second portions, the third portion having a third diameter less than the first diameter or the second diameter;
a catheter shaft for supporting the inflatable balloon, the catheter shaft including a first inflation lumen in communication with a first opening associated with the first portion of the inflatable balloon and a second inflation lumen in communication with the second portion of the inflatable balloon; and
a first connector adapted for supplying fluid to one or both of the first and second inflation lumens.

11. The medical apparatus of claim 10, wherein the third portion of the balloon is attached to the shaft to block fluid communication between the first portion of the balloon and the second portion of the balloon.

12. The medical apparatus of claim 10, wherein the first connector is adapted for supplying fluid to the first inflation lumen and further including a second connector adapted for supplying fluid to the second inflation lumen.

13. The medical apparatus of claim 10, wherein the first connector is in communication with a third inflation lumen in communication with the first and second inflation lumens, and further including a valve for controlling fluid flow from the third inflation lumen to the first and second inflation lumens.

14. The medical apparatus of claim 10, wherein the balloon is compliant or semi-compliant.

15. An implant in combination with the medical apparatus of claim 10.

16. A method of inflating a medical balloon inserted in a vessel, comprising:
inflating spaced apart portions of the medical balloon while restraining an intermediate portion of the medical balloon to provide the inflated medical balloon with an hourglass shape within the vessel.

17. The method of claim 16, wherein the inflating step comprises supplying inflation fluid simultaneously to first and second end portions of the medical balloon.

18. The method of claim 16, further comprising the step of supplying inflation fluid independently to first and second end portions of the medical balloon after the inflating step.

19. The method of claim 16, wherein the inflating step comprises supplying inflation fluid independently to first and second end portions of the medical balloon.

20. The method of claim 16, further including the step of delivering an implant to the vessel via the medical balloon.
